(19) United States
(12) Patent Application Publication
(10) Pub. No.: US 2023/0368447 A1
(43) Pub. Date: Nov. 16, 2023

(54) KNEE TRAJECTORY INFORMATION GENERATION DEVICE, KNEE TRAJECTORY INFORMATION GENERATION METHOD, AND RECORDING MEDIUM

(71) Applicant: NEC Corporation, Tokyo (JP)

(72) Inventors: Kazuki IHARA, Tokyo (JP); Kenichiro FUKUSHI, Tokyo (JP); Zhenwei WANG, Tokyo (JP); Hiroshi KAJITANI, Tokyo (JP); Fumiyuki NIHEY, Tokyo (JP); Chenhui HUANG, Tokyo (JP); Yoshitaka NOZAKI, Tokyo (JP); Kentaro NAKAHARA, Tokyo (JP)

(73) Assignee: NEC Corporation, Tokyo (JP)

(21) Appl. No.: 18/139,753

(22) Filed: Apr. 26, 2023

(30) Foreign Application Priority Data
May 11, 2022 (JP) 2022-077836

(51) Int. Cl. G06T 11/60; A61B 5/11; A61B 5/00

(52) U.S. Cl. CPC G06T 11/60; A61B 5/1122; A61B 5/112; A61B 5/1127; A61B 5/743; A61B 2562/0219; G06T 2210/41

(57) ABSTRACT

A knee trajectory information generation device which includes an acquisition unit that acquires walking data including time-series data of a foot position and a knee position of a subject, a first calculation unit that calculates a first movement route connecting a start point and an end point of a gait cycle by using time-series data of the foot position included in the walking data, a second calculation unit that calculates a second movement route corresponding to a trajectory of the knee position between the start point and the end point of the gait cycle by using time-series data of the knee position included in the walking data, an information generation unit that calculates a difference between the first movement route and the second movement route and generate knee trajectory information including visual information corresponding to the calculated difference, and an output unit that outputs the generated knee trajectory information.

This application is based upon and claims the benefit of priority from Japanese Patent Application No. 2022-077836, filed on May 11, 2022, the disclosure of which is incorporated herein in its entirety by reference.

### TECHNICAL FIELD

The present disclosure relates to a knee trajectory information generation device and the like that generate information regarding a knee trajectory during walking.

### BACKGROUND ART

With increasing interest in healthcare, information according to features (also referred to as gait) included in a gait pattern has been attracting. Utilizing the information corresponding to the gait, healthcare services can be provided for various symptoms that people suffer from. Information indicating the motion of the knee during walking is useful for diagnosis of knee osteoarthritis or the like. In particular, if the behavior of the knee in the left-right direction can be grasped, early detection and prevention of knee osteoarthritis can be achieved.

Patent Literature 1 (JP 2017-023436 A) discloses a walking analysis system that calculates a walking parameter used for evaluation of walking motion of a subject. The system of Patent Literature 1 measures acceleration and angular velocity using a triaxial acceleration sensor and a triaxial angular velocity sensor attached to a lower limb portion of a subject. The system of Patent Literature 1 calculates the posture of the lower limb portion during walking based on the measured acceleration and angular velocity. The system of Patent Literature 1 constructs a three-dimensional model including a motion trajectory of a joint by connecting lower limb portions in the calculated posture to each other. The system of Patent Literature 1 calculates an angle formed by an acceleration vector of a joint in a sagittal plane at the time of heel strike with respect to a motion trajectory as a walking parameter.

Patent Literature 2 (JP 2021-176347 A) discloses a motion information display device that displays a periodic motion of an organism. The device of Patent Literature 2 acquires motion information of a target organism from a moving image of the target organism. The device of Patent Literature 2 corrects the influence of the translational movement of the specific part according to the reference of the motion information. The device of Patent Literature 2 stores the corrected position information of the specific part over a plurality of frames of a moving image. The device of Patent Literature 2 superimposes the trajectory of the specific part on the moving image, and displays the moving image on which the trajectory is superimposed.

In the method of Patent Literature 1, an angle formed by an acceleration vector of a joint at the time of heel strike with respect to a motion trajectory (corresponding to an angle of a knee) is calculated as a walking parameter using a triaxial acceleration sensor and a triaxial angular velocity sensor attached to a lower limb portion. However, in the method of Patent Literature 1, it is not possible to generate information with which the behavior of the knee in the left-right direction can be grasped.

According to the method of Patent Literature 2, the trajectory of the ankle joint as seen from a lateral viewpoint can be displayed in two dimensions. based on the moving image of the target organism. However, in the method of Patent Literature 2, it is not possible to display information with which the behavior of the knee viewed from the front viewpoint can be grasped.

An object of the present disclosure is to provide a knee trajectory information generation device and the like capable of generating information regarding a knee trajectory including a knee behavior in a left-right direction.

### SUMMARY

A knee trajectory information generation device according to one aspect of the present disclosure includes: an acquisition unit configured to acquire walking data including time-series data of a foot position and a knee position of a subject; a first calculation unit configured to calculate a first movement route connecting a start point and an end point of a gait cycle by using time-series data of the foot position included in the walking data; a second calculation unit configured to calculate a second movement route corresponding to a trajectory of the knee position between the start point and the end point of the gait cycle by using time-series data of the knee position included in the walking data; an information generation unit configured to calculate a difference between the first movement route and the second movement route and generate knee trajectory information including visual information corresponding to the calculated difference; and an output unit configured to output the generated knee trajectory information.

In a knee trajectory information generation method according to one aspect of the present disclosure, walking data including time-series data of a foot position and a knee position of a subject is acquired; a first movement route connecting a start point and an end point of a gait cycle is calculated by using time-series data of the foot position included in the walking data; a second movement route corresponding to a trajectory of the knee position between the start point and the end point of the gait cycle is calculated by using time-series data of the knee position included in the walking data; a difference between the first movement route and the second movement route is calculated and knee trajectory information including visual information corresponding to the calculated difference is generated; and the generated knee trajectory information is output.

A program according to one aspect of the present disclosure causes a computer to execute: acquiring walking data including time-series data of a foot position and a knee position of a subject; calculating a first movement route connecting a start point and an end point of a gait cycle by using time-series data of the foot position included in the walking data; calculating a second movement route corresponding to a trajectory of the knee position between the start point and the end point of the gait cycle by using time-series data of the knee position included in the walking data; calculating a difference between the first movement route and the second movement route and generate knee trajectory information including visual information corresponding to the calculated difference; and outputting the generated knee trajectory information.

### BRIEF DESCRIPTION OF THE DRAWINGS

Exemplary features and advantages of the present invention will become apparent from the following detailed description when taken with the accompanying drawings in which:

FIG. 1 is a block diagram illustrating an example of a configuration of a knee trajectory information generation device according to a first example embodiment;

FIG. 2 is a conceptual diagram for explaining an example of a walking event in the first example embodiment;

FIG. 3 is a conceptual diagram for explaining an example of a human body surface in the first example embodiment;

FIG. 4 is a graph illustrating an example of time-series data of a difference between a first movement route and a second movement route calculated by the knee trajectory information generation device according to the first example embodiment;

FIG. 5 is a graph illustrating another example of time-series data of a difference between the first movement route and the second movement route calculated by the knee trajectory information generation device according to the first example embodiment;

FIG. 6 is a conceptual diagram for explaining a first example of visual information generated by the knee trajectory information generation device according to the first example embodiment;

FIG. 7 is a conceptual diagram for explaining a second example of the visual information generated by the knee trajectory information generation device according to the first example embodiment;

FIG. 8 is a conceptual diagram for explaining a third example of the visual information generated by the knee trajectory information generation device according to the first example embodiment;

FIG. 9 is a conceptual diagram for explaining a fourth example of the visual information generated by the knee trajectory information generation device according to the first example embodiment;

FIG. 10 is a conceptual diagram for explaining a fifth example of the visual information generated by the knee trajectory information generation device according to the first example embodiment;

FIG. 11 is a conceptual diagram for explaining the fifth example of the visual information generated by the knee trajectory information generation device according to the first example embodiment;

FIG. 12 is a conceptual diagram for explaining a sixth example of the visual information generated by the knee trajectory information generation device according to the first example embodiment;

FIG. 13 is a flowchart for explaining an example of the operation of the knee trajectory information generation device according to the first example embodiment;

FIG. 14 is a conceptual diagram for explaining Application Example 1-1 according to the first example embodiment;

FIG. 15 is a conceptual diagram for explaining the Application Example 1-1 according to the first example embodiment;

FIG. 16 is a conceptual diagram for explaining Application Example 1-2 according to the first example embodiment;

FIG. 17 is a conceptual diagram for explaining the Application Example 1-2 according to the first example embodiment;

FIG. 18 is a conceptual diagram for explaining Application Example 1-3 according to the first example embodiment;

FIG. 19 is a conceptual diagram for explaining the Application Example 1-3 according to the first example embodiment;

FIG. 20 is a conceptual diagram for explaining the Application Example 1-3 according to the first example embodiment;

FIG. 21 is a block diagram illustrating an example of a configuration of a knee trajectory information generation device according to a second example embodiment; and

FIG. 22 is a block diagram illustrating an example of a hardware configuration that executes processing according to each example embodiment.

### EXAMPLE EMBODIMENT

Example embodiments of the present invention will be described below with reference to the drawings. In the following example embodiments, technically preferable limitations are imposed to carry out the present invention, but the scope of this invention is not limited to the following description. In all drawings used to describe the following example embodiments, the same reference numerals denote similar parts unless otherwise specified. In addition, in the following example embodiments, a repetitive description of similar configurations or arrangements and operations may be omitted.

### First Example Embodiment

First, a configuration of a knee trajectory information generation device according to a first example embodiment will be described with reference to the drawings. The knee trajectory information generation device according to the present example embodiment acquires walking data measured according to the walking of a subject. The walking data includes foot position data and knee position data. The knee trajectory information generation device according to the present example embodiment generates information (also referred to as knee trajectory information) regarding the knee trajectory indicating the motion of the knee using the walking data.

In the present example embodiment, the center position of the knee is referred to as a knee position. The knee position may be shifted from the center position of the knee as long as the verification of the knee trajectory is not affected. In the present example embodiment, the center position of the foot is referred to as a foot position. The foot position may be shifted from the center position of the foot as long as the verification of the knee trajectory is not affected.

(Configuration)

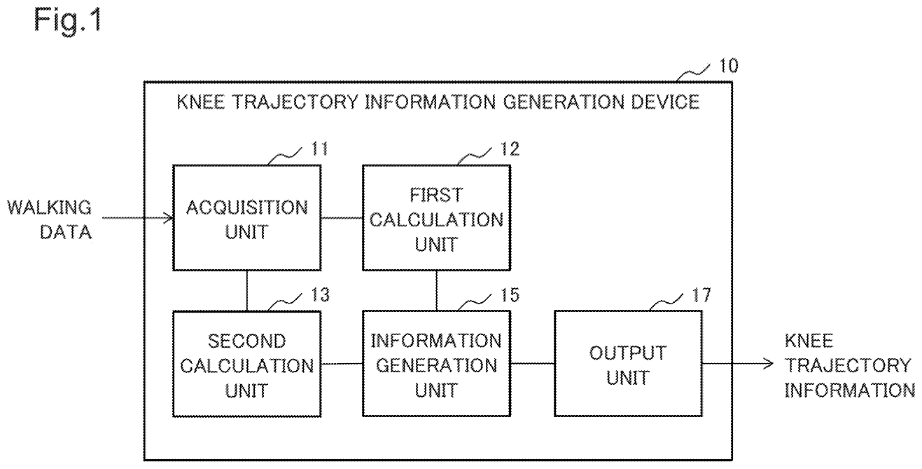

FIG. 1 is a block diagram illustrating a configuration of a knee trajectory information generation device 10 according to the present example embodiment. The knee trajectory information generation device 10 includes an acquisition unit 11, a first calculation unit 12, a second calculation unit 13, an information generation unit 15, and an output unit 17.

The acquisition unit 11 acquires the walking data of the subject. The walking data includes foot position data and knee position data of the subject. The foot position data is a time change of a three-dimensional foot position. The knee position data is a time change of a three-dimensional knee position. A method for measuring the foot position data and the knee position data is not particularly limited.

For example, the foot position data and the knee position data are measured by motion capture. In the motion capture, a marker is attached to each part of the subject's body. For example, the marker is attached to a site including a foot and a knee. A walking subject is photographed with a camera, and a foot position and a knee position are measured according to a position of a marker in the photographed image (video). According to the motion capture, since the foot position and the knee position can be directly measured, highly accurate foot position data and knee position data can be obtained.

For example, the foot position data and the knee position data are measured by analyzing an image (video) captured by the camera. By using software such as OpenPose, the foot position data and the knee position data are measured by calculating the foot position and the knee position based on the positions of the skeleton or the joints detected from the person in the image.

For example, the foot position data and the knee position data are measured using acceleration and angular velocity measured by an inertial sensor attached to the knee. When the inertial sensor is used, the foot position and the knee position can be calculated by integrating the acceleration and the angular velocity. For example, the foot position data and the knee position data may be measured using a smart apparel in which an inertial sensor is attached to each part of the entire body.

For example, the foot position data is measured according to a foot position measured using an inertial sensor installed on the footwear. In this case, the knee position data is estimated according to the measured foot position.

The acquisition unit 11 acquires the walking data in the predetermined walking section. For example, the predetermined walking section is one gait cycle. The predetermined walking section may be a plurality of gait cycles. In the following description, a period from the landing of the heel of the right foot to the landing of the heel of the right foot again is defined as one gait cycle of the right foot. Similarly, a period from the landing of the heel of the left foot to the landing of the heel of the left foot again is defined as one gait cycle of the left foot. The event in which the heel lands is called heel strike. The start point and the end point of the gait cycle may be set to timings of events other than heel landing.

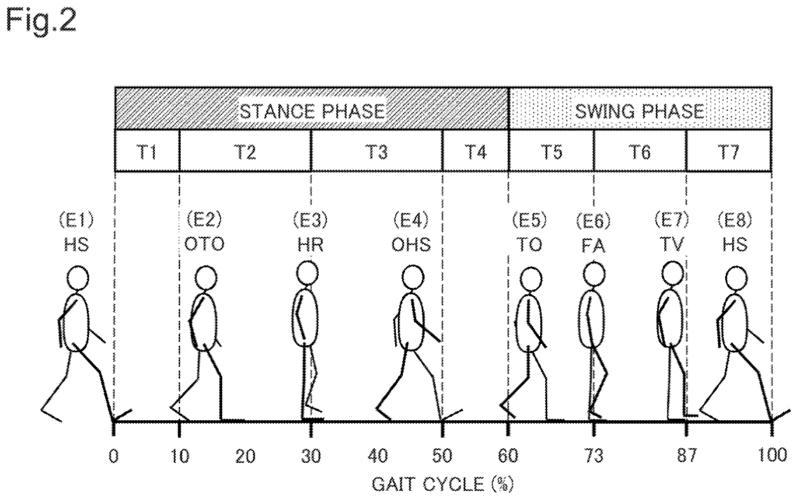

FIG. 2 is a conceptual diagram for explaining a walking event detected in one gait cycle with the right foot as a reference. The horizontal axis of FIG. 2 is a gait cycle normalized with one gait cycle of the right foot as 100%. A time point at which the heel of the right foot lands on the ground is defined as a starting point (0%), and a time point at which the heel of the right foot next lands on the ground is defined as an end point (100%). Each of the plurality of timings included in one gait cycle is referred to as a walking phase. The one gait cycle of one foot is roughly divided into a stance phase in which at least a part of the back side of the foot is in contact with the ground and a swing phase in which the back side of the foot is separated from the ground. In the example of FIG. 2, the gait cycle is normalized such that the stance phase occupies 60% and the swing phase occupies 40%. The stance phase is subdivided into an initial stance period T1, a mid-stance period T2, a terminal stance period T3, and a pre-swing period T4. The swing phase is subdivided into an initial swing period T5, a mid-swing period T6, and a terminal swing period T7. The walking waveform in one gait cycle may not start from the time point when the heel lands on the ground. For example, the starting point of the walking waveform in one gait cycle may be set at a central time point of the stance phase or the like.

A walking event E1 represents a heel strike (HS) at the beginning of one gait cycle. The heel strike is an event in which the heel of the right foot, which has been separated from the ground in the swing phase, lands on the ground. A walking event E2 represents an opposite toe off (ONO). The opposite toe off is an event in which the toe of the left foot is separated from the ground in a state where the ground contact surface of the sole of the right foot is in contact with the ground. A walking event E3 represents a heel rise (HR). The heel rise is an event in which the heel of the right foot rises while the ground contact surface of the sole of the right foot is in contact with the ground. A walking event E4 represents an opposite heel strike (OHS). The opposite heel strike is an event in which the heel of the left foot, which has been separated from the ground in the swing phase of the left foot, lands on the ground. A walking event E5 represents a toe off (TO). The toe off is an event in which the toe of the right foot is off the ground in a state where the ground contact surface of the sole of the left foot is in contact with the ground. A walking event E6 represents a foot adjacent (FA). The foot adjacent is an event in which the left foot and the right foot cross each other in a state where the ground contact surface of the sole of the left foot is grounded. A walking event E7 represents a tibia vertical (TV). The tibia vertical is an event in which the tibia of the right foot is substantially perpendicular to the ground while the sole of the left foot is grounded. A walking event E8 represents the heel strike (HS) at the end of one gait cycle. The walking event E8 corresponds to the end point of the gait cycle starting from the walking event E1 and corresponds to the starting point of the next gait cycle.

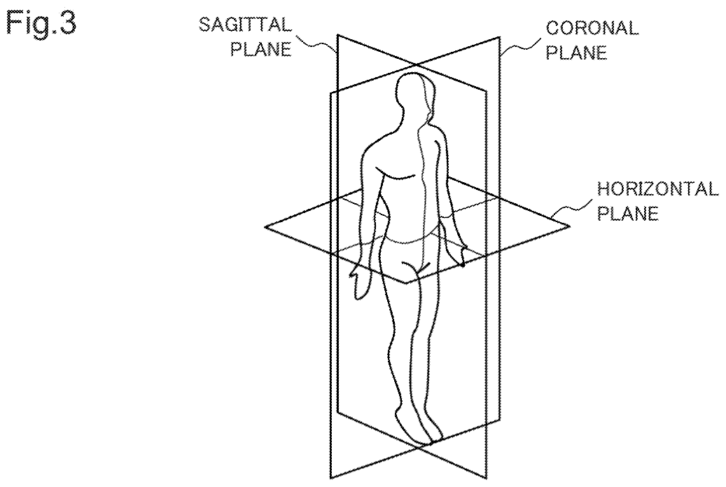

FIG. 3 is a conceptual diagram for explaining a surface (also referred to as a human body surface) set for the human body. In the present example embodiment, a sagittal plane dividing the body into left and right, a coronal plane dividing the body into front and rear, and a horizontal plane dividing the body horizontally are defined. In the present example embodiment, rotation in the sagittal plane with the x axis as the rotation axis is defined as roll, rotation in the coronal plane with the y axis as the rotation axis is defined as pitch, and rotation in the horizontal plane with the z axis as the rotation axis is defined as yaw. A rotation angle in a sagittal plane with the x axis as a rotation axis is defined as a roll angle, a rotation angle in a coronal plane with the y axis as a rotation axis is defined as a pitch angle, and a rotation angle in a horizontal plane with the z axis as a rotation axis is defined as a yaw angle. In the present example embodiment, in the coronal plane, the right side is defined as positive for the right foot, and the left side is defined as positive for the left foot.

The first calculation unit 12 acquires foot position data in a predetermined gait cycle. The foot position data includes the foot positions at the start point and the end point for each gait cycle. In the present example embodiment, the point of time of the continuous heel strike is set as the start point/end point of each gait cycle. For example, the foot position data includes the foot position in one gait cycle with the heel strike as a start point/end point. For example, the foot position data includes the foot position in the horizontal plane at the time point of the start point (heel strike) and the foot position in the horizontal plane at the time point of the end point (heel strike) with respect to the predetermined gait cycle.

The first calculation unit 12 calculates a walking movement route (also referred to as a first movement route) connecting a start point and an end point included in the foot position data. In the present example embodiment, a straight line connecting the foot position on the horizontal plane at the start point (heel strike) and the foot position on the horizontal plane at the end point (heel strike) is defined as the first movement route.

The second calculation unit 13 acquires knee position data in a predetermined gait cycle. The knee position data includes the knee positions at the start point and the end point for each gait cycle. For example, the knee position data includes a knee position in one gait cycle with heel strike as a start point/end point. The knee position data includes the knee position in the horizontal plane at the time point of the start point (heel strike) and the knee position in the horizontal plane at the time point of the end point (heel strike) with respect to the predetermined gait cycle.

The second calculation unit 13 calculates a knee movement route (also referred to as a second movement route) connecting the start point and the end point included in the knee position data. In the present example embodiment, a curve connecting the knee position in the horizontal plane at the time point of the start point (heel strike) and the knee position in the horizontal plane at the time point of the end point (heel strike) is defined as the second movement route. The second movement route corresponds to a knee trajectory.

The information generation unit 15 acquires the first movement route and the second movement route in a predetermined gait cycle. The information generation unit 15 calculates a difference between the first movement route and the second movement route for each predetermined gait cycle. In the present example embodiment, the information generation unit 15 calculates a difference between the first movement route and the second movement route in the horizontal plane. The information generation unit 15 calculates a difference between the first movement route and the second movement route in the horizontal plane in association with the walking phase included in a predetermined walking section. The difference between the first movement route and the second movement route is positive on the right side with respect to the right foot. The difference between the first movement route and the second movement route is positive on the left side with respect to the left foot. For example, the information generation unit 15 calculates a difference between the first movement route and the second movement route in the horizontal plane for one gait cycle. For example, the information generation unit 15 associates the calculated difference with a position (traveling direction position) in the sagittal plane for one gait cycle. For example, the traveling direction position corresponding to one gait cycle is converted into a gait cycle and associated with the difference.

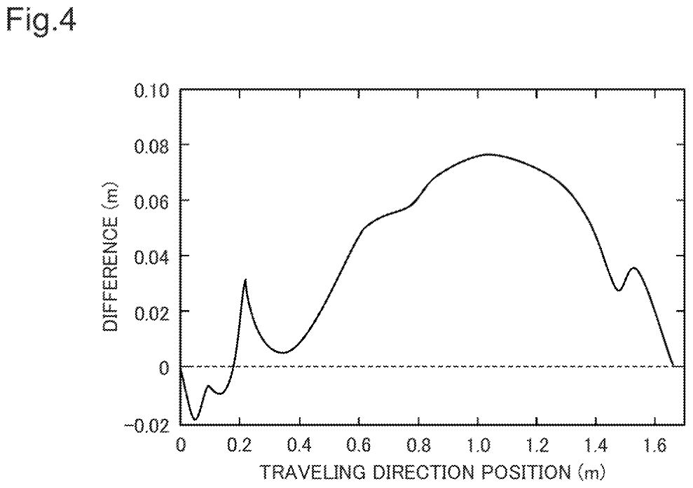

FIG. 4 is a graph in which a difference between the first movement route and the second movement route in the horizontal plane for one gait cycle is associated with a traveling direction position for one gait cycle. FIG. 4 is a difference regarding the right foot. When the difference is positive, the knee position is shifted rightward from the first movement route. That is, when the difference is positive, the knee position is shifted outward from the center of the body. On the other hand, when the difference is negative, the knee position is shifted leftward from the first movement route. That is, when the difference is negative, the knee is shifted inward from the center of the body. The larger the absolute value of the difference, the larger the amount of deviation of the knee position from the center of the body.

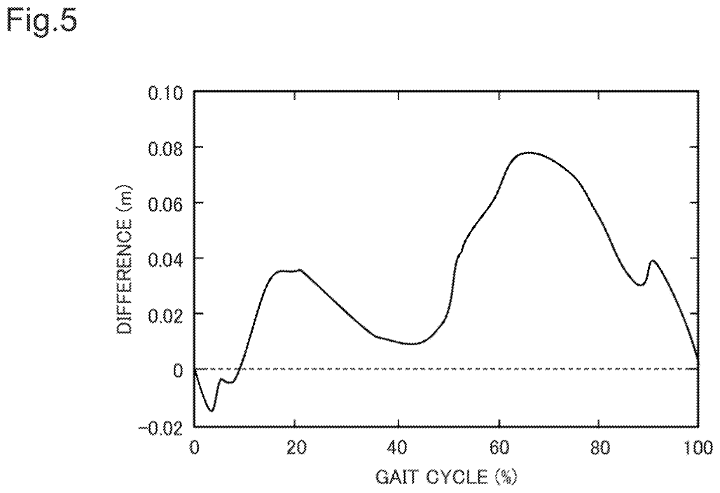

FIG. 5 is a graph in which a difference between the first movement route and the second movement route in the horizontal plane for one gait cycle is associated with the gait cycle. FIG. 5 is a graph obtained by normalizing the horizontal axis (traveling direction position) of the graph of FIG. 4 to the gait cycle. As illustrated in FIG. 5, when the horizontal axis of the time-series data of the difference is converted into the gait cycle, the variation of the knee trajectory over a plurality of gait cycles can be compared for each walking phase.

The knee trajectory information generation device 10 generates knee trajectory information according to a difference between the first movement route and the second movement route. The knee trajectory information includes visual information expressing a knee trajectory according to walking of the subject. For example, the visual information on the knee trajectory is an arrow indicating the direction and magnitude of the difference between the first movement route and the second movement route. For example, the visual information on the knee trajectory is a graph in which time-series data of differences between the first movement route and the second movement route over a plurality of gait cycles is superimposed. For example, the visual information on the knee trajectory is a mark (also referred to as a first sign) indicating the height of the knee. For example, the visual information on the knee trajectory is an arrow (also referred to as a second sign) indicating the direction and magnitude of the difference between the first movement route and the second movement route. For example, the visual information on the knee trajectory is a sign obtained by combining the first sign and the second sign. For example, the sign is displayed in accordance with the video of the walking subject (character). The knee trajectory information is not particularly limited as long as it includes visual information regarding the knee trajectory.

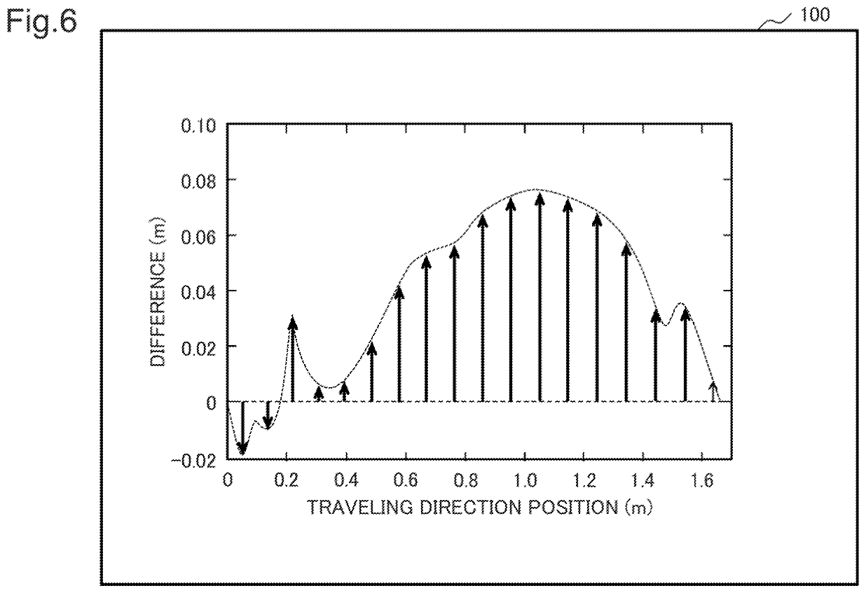

FIG. 6 is a first example of visual information on the knee trajectory. FIG. 6 is an example in which arrows representing the direction and magnitude of the difference between the first movement route and the second movement route are displayed on a screen 100 in association with the traveling direction position. The direction of the arrow indicates the direction of the difference. The length of the arrow indicates the magnitude of the difference. The arrow is set to a length corresponding to the magnitude of the difference. For example, the arrow is set to have a length that matches the magnitude of the difference. For example, the arrow is set to a length obtained by multiplying the magnitude of the difference. Instead of displaying the time-series data of the difference between the first movement route and the second movement route, only the arrows representing the direction and the magnitude of the difference may be displayed. According to the visual information of FIG. 6, the change in the knee trajectory can be intuitively grasped by the direction and length of the arrow associated with the traveling direction position.

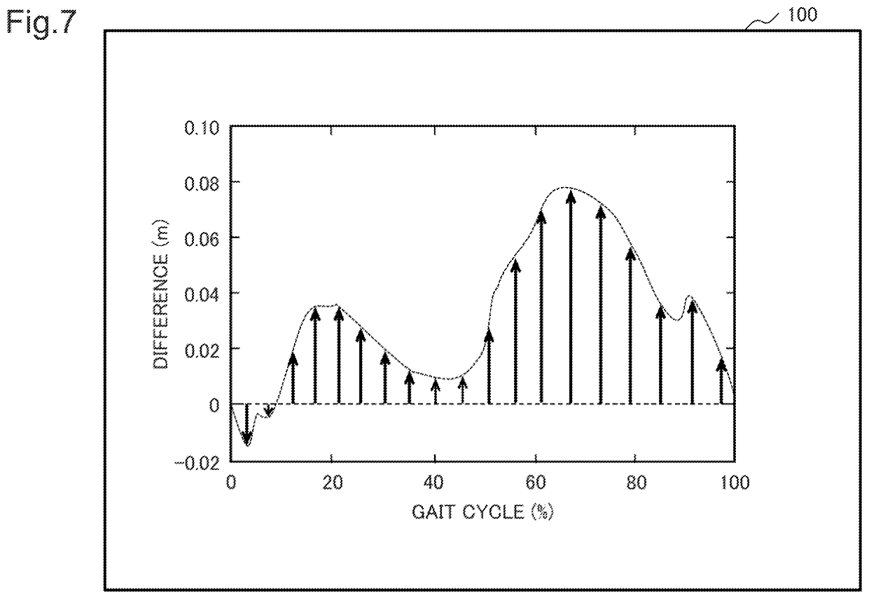

FIG. 7 is a second example of the visual information on the knee trajectory. FIG. 7 is an example in which arrows representing the direction and magnitude of the difference between the first movement route and the second movement route are displayed on the screen 100 in association with the gait cycle. FIG. 7 is a graph obtained by converting the horizontal axis of the graph of FIG. 6 into a gait cycle. Instead of displaying the time-series data of the difference between the first movement route and the second movement route, only the arrows representing the direction and the magnitude of the difference may be displayed. According to the visual information of FIG. 7, it is possible to intuitively grasp the change in the knee trajectory according to the walking phase by the direction and magnitude of the arrow associated with the gait cycle.

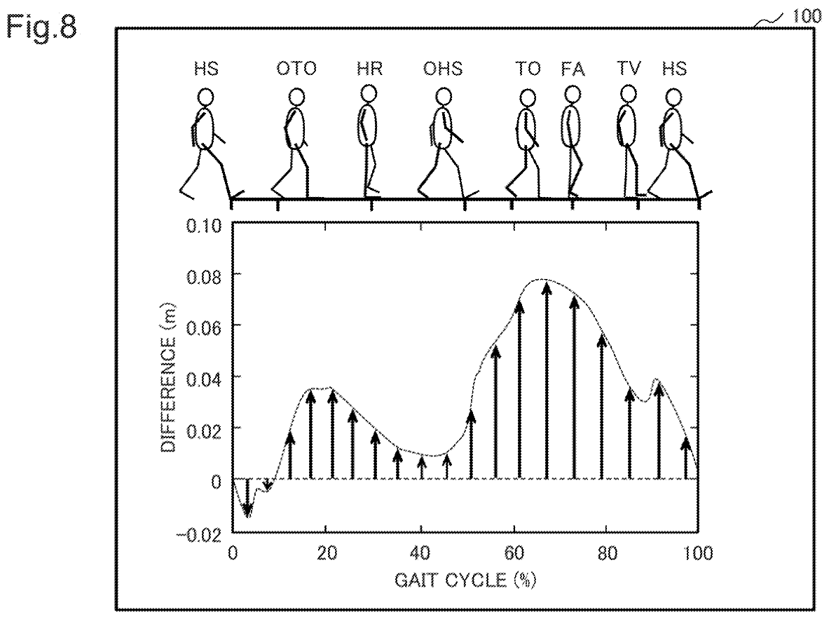

FIG. 8 is a third example of the visual information on the knee trajectory. In the example of FIG. 8, an arrow indicating the direction and magnitude of the difference between the first movement route and the second movement route is displayed on the screen 100 in association with the gait cycle. The graph of FIG. 8 is the same as the graph of FIG. 7. In the example of FIG. 8, a character indicating a walking state according to the walking event is displayed on the screen 100 in association with the gait cycle. The character may be a conceptual character imitating walking of a person or an image of a walking person. Instead of displaying the time-series data of the difference between the first movement route and the second movement route, only the arrows representing the direction and the magnitude of the difference may be displayed. According to the visual information of FIG. 8, it is possible to intuitively grasp the change in the knee trajectory according to the walking event by the direction and magnitude of the arrow associated with the gait cycle and the character indicating the walking state according to the walking event.

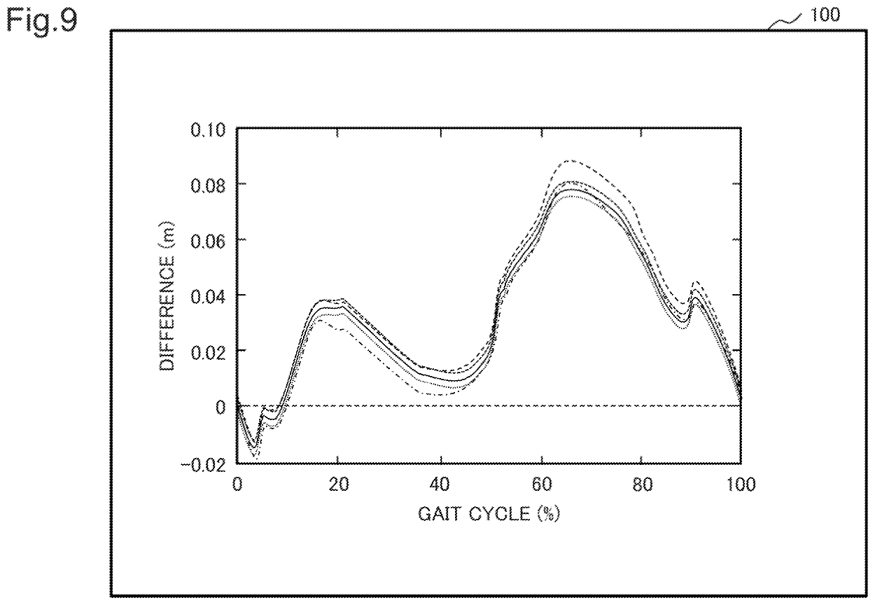

FIG. 9 is a fourth example of the visual information on the knee trajectory. FIG. 9 is an example in which time-series data of differences between the first movement route and the second movement route over a plurality of gait cycles is displayed to be superimposed on the screen 100. According to the visual information of FIG. 9, it is possible to intuitively grasp the variation of the knee trajectory by comparing the time-series data of the differences over the plurality of gait cycles. For example, statistical values such as an arithmetic mean, a geometric mean, a variance, and a standard deviation regarding the knee trajectory (knee position) over a plurality of gait cycles may be derived. By using an average value such as an arithmetic mean or a geometric mean of the knee trajectories (knee positions) regarding a plurality of gait cycles, the motion of the knee in the plurality of gait cycles can be grasped on average. By using the variance, standard deviation, and the like of the knee trajectory (knee position) regarding a plurality of gait cycles, it is possible to grasp the fluctuation of the knee motion in the plurality of gait cycles.

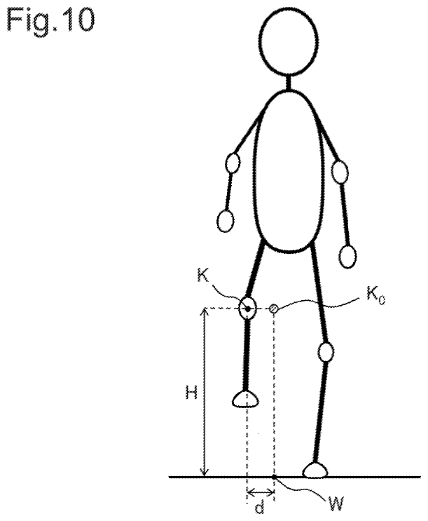

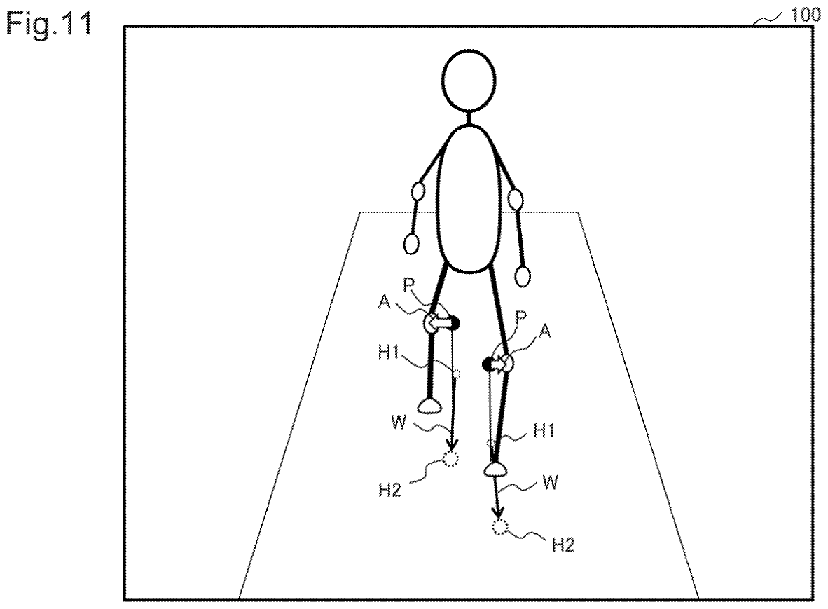

FIGS. 10 to 11 are conceptual diagrams for explaining a fifth example of the visual information regarding the knee trajectory. FIG. 10 is a conceptual diagram of a walking person (character) as viewed from the front. The person (character) in FIG. 10 is in a state where the left foot is a support leg and the right foot is separated from the ground. FIG. 10 illustrates a knee position K in the coronal plane, a knee height H in the coronal plane, a first movement route W, a difference d between the first movement route W and the second movement route (knee position K), and a position K₀ of the knee height H immediately above the first movement route W.

FIG. 11 is a conceptual diagram illustrating an example in which visual information regarding a knee trajectory is displayed on the screen 100 in accordance with a video indicating a state in which a person (character) walks. FIG. 11 illustrates, for each of the left and right feet, the position of the continuous heel strike (start point H1 and end point H2) and the first movement route W connecting the start point H1 and the end point H2. In FIG. 11, a pin P having the head at the position of the knee height H (position K₀ in FIG. 10) immediately above the first movement route W is raised on the first movement route W. The pin P is raised on the first movement route W in association with the walking phase related to each of the left and right legs. The position where the pin P is raised may be shifted from the first movement route W. The first movement route W on which the pin P is raised may be shifted from the straight line connecting the start point H1 and the end point H2. In the example of FIG. 11, the straight line indicating the first movement route W includes an arrowhead indicating the traveling direction. The straight line indicating the first movement route W may not include the arrowhead indicating the traveling direction.

In the example of FIG. 11, an arrow A indicating the direction and magnitude of the difference between the first movement route W and the second movement route (the knee position K in FIG. 10) is displayed with the head of the pin P as a start point. Similarly to the arrows in FIGS. 7 to 8, the direction of the arrow A indicates the direction of the difference between the first movement route and the second movement route. The length of the arrow A indicates the magnitude of the difference. For example, the arrow A is set to have a length that matches the magnitude of the difference. For example, the arrow A is set to have a length obtained by multiplying the magnitude of the difference. The direction and length of the arrow A are changed in conjunction with the walking phase of the person (character). For example, not the entire body of the person (character) but only a portion below the waist (lower body) may be displayed on the video. According to the visual information of FIG. 11, it is possible to intuitively grasp the variation of the knee trajectory according to walking according to the movement of the pin P and the arrow A that vary according to the video of the person (character).

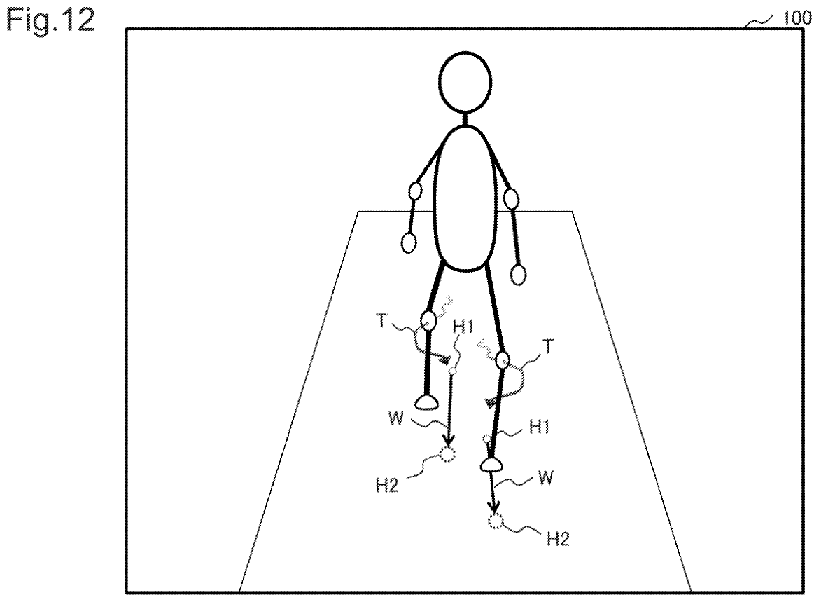

FIG. 12 is a conceptual diagram illustrating a sixth example in which visual information regarding a knee trajectory is displayed on the screen 100 in accordance with a video indicating a state in which a person (character) walks. FIG. 12 illustrates, for each of the left and right feet, the position of the continuous heel strike (start point H1 and end point H2) and the first movement route W connecting the start point H1 and the end point H2. In FIG. 12, the knee trajectory T is displayed in conjunction with the motion of the knee. A knee trajectory T corresponds to time-series data of a difference between the first movement route and the second movement route in FIGS. 7 to 8. In the example of FIG. 12, the knee trajectory T is displayed in association with the knee position. The position where the knee trajectory is displayed may be shifted from the knee position. For example, the knee position in the knee trajectory T is changed in conjunction with the walking phase of the person (character). According to the visual information of FIG. 12, it is possible to intuitively grasp the variation of the knee trajectory according to walking according to the knee trajectory T displayed in accordance with the video of the person (character).

The output unit 17 outputs the knee trajectory information generated by the information generation unit 15. For example, the output unit 17 outputs the knee trajectory information to a terminal device having a screen. The knee trajectory information output to the terminal device is displayed on the screen of the terminal device. For example, the output unit 17 displays the knee trajectory information on the screen of the mobile terminal of the subject (user). For example, the output unit 17 displays the knee trajectory information on a screen of a terminal device used by an expert such as a doctor, a physical therapist, or a care worker who verifies the physical condition of the subject. The expert can give a diagnosis or advice according to the knee trajectory information displayed on the screen of the terminal device to the subject. For example, the output unit 17 may output the knee trajectory information to an external system or the like that uses the knee trajectory information. The use of the knee trajectory information output from the output unit 17 is not particularly limited.

For example, the knee trajectory information generation device 10 is connected to an external system or the like built in a cloud or a server via a mobile terminal (not illustrated) carried by a subject (user). The mobile terminal is a portable communication device. For example, the mobile terminal is a portable communication device having a communication function, such as a smartphone, a smart watch, or a mobile phone.

For example, the knee trajectory information generation device 10 is connected to a terminal device (not illustrated) used by a person who verifies the physical condition of a subject (user). Software for processing the knee trajectory information and displaying an image according to the knee trajectory information is installed in the terminal device. For example, the terminal device is an information processing device such as a stationary personal computer, a notebook personal computer, a tablet, or a mobile terminal. The terminal device may be a dedicated terminal that processes the knee trajectory information.

For example, the knee trajectory information generation device 10 is connected to a mobile terminal or a terminal device via a wire such as a cable. For example, the knee trajectory information generation device 10 is connected to a mobile terminal or a terminal device via wireless communication. For example, the knee trajectory information generation device 10 is connected to a mobile terminal or a terminal device via a wireless communication function (not illustrated) conforming to a standard such as Bluetooth (registered trademark) or WiFi (registered trademark). The communication function of the knee trajectory information generation device 10 may conform to a standard other than Bluetooth (registered trademark) or WiFi (registered trademark). The knee trajectory information may be used by an application installed in a mobile terminal or a terminal device. In that case, the mobile terminal or the terminal device executes processing using the knee trajectory information by application software or the like installed in the device. The knee trajectory information generation device 10 may be mounted on a mobile terminal or a terminal device.

(Operation)

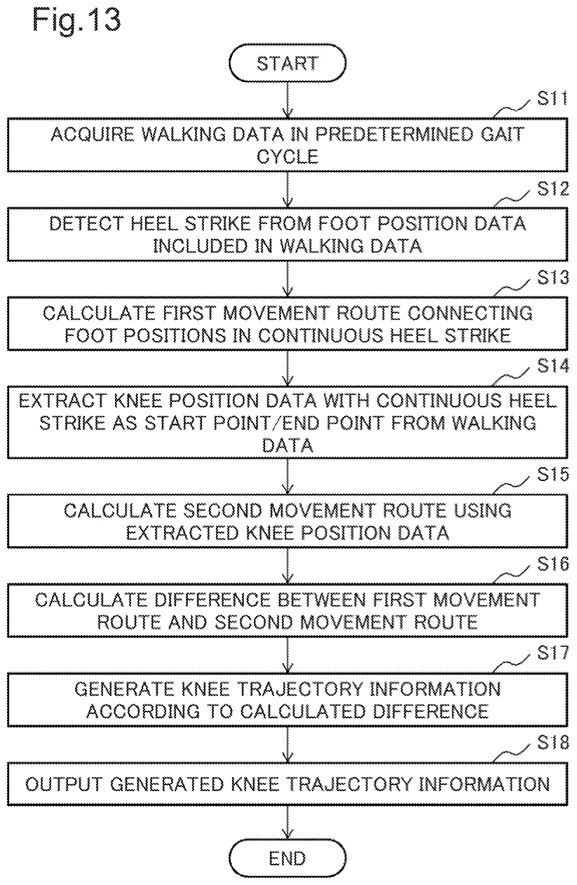

Next, an example of the operation of the knee trajectory information generation device 10 will be described with reference to the drawings. FIG. 13 is a flowchart for explaining an example of the operation of the knee trajectory information generation device 10. In the description along the flowchart of FIG. 13, the knee trajectory information generation device 10 will be described as an operation subject.

In FIG. 13, first, the knee trajectory information generation device 10 acquires walking data in a predetermined gait cycle (step S11). The walking data includes time-series data of a foot position (foot position data) and time-series data of a knee position (knee position data). For example, the knee trajectory information generation device 10 acquires walking data of one gait cycle. The knee trajectory information generation device 10 may acquire walking data over a plurality of gait cycles.

Next, the knee trajectory information generation device 10 detects heel strike from the foot position data included in the walking data (step S12). The knee trajectory information generation device 10 detects heel strike corresponding to the start point/end point of the foot position data in one gait cycle. In the case of a plurality of gait cycles, the knee trajectory information generation device 10 detects heel strike corresponding to the start point/end point of the foot position data for each gait cycle.

Next, the knee trajectory information generation device 10 calculates a first movement route connecting the foot positions in the continuous heel strike (step S13). The first movement route is a straight line serving as a reference of the knee trajectory.

Next, the knee trajectory information generation device 10 extracts knee position data with continuous heel strike as the start point/end point from the walking data (step S14). In the case of a plurality of gait cycles, the knee trajectory information generation device 10 extracts, from the walking data, knee position data for each gait cycle with a continuous heel strike as the start point/end point.

Next, the knee trajectory information generation device 10 calculates the second movement route using the extracted knee position data (step S15). The second movement route is a curve corresponding to the knee trajectory. In the case of a plurality of gait cycles, the knee trajectory information generation device 10 calculates the second movement route for each gait cycle.

Next, the knee trajectory information generation device 10 calculates a difference between the second movement route and the first movement route (step S16). The difference between the second movement route and the first movement route is a curve corresponding to the knee trajectory based on the first movement route. In the case of a plurality of gait cycles, the knee trajectory information generation device 10 calculates a difference for each gait cycle.

Next, the knee trajectory information generation device 10 generates the knee trajectory information according to the calculated difference (step S17). The knee trajectory information includes the visual information regarding the knee trajectory. For example, the knee trajectory information generation device 10 generates the visual information according to any one of the first to sixth examples (FIGS. 6 to 12) described above.

Next, the knee trajectory information generation device 10 outputs the generated knee trajectory information (step S18). The knee trajectory information generation device 10 outputs the knee trajectory information including the visual information regarding the knee trajectory. The visual information included in the output knee trajectory information is displayed on a screen of a terminal device (not illustrated) or the like used by the user who uses the knee trajectory information.

### Application Example

Next, an application example of the knee trajectory information generation device 10 will be described with reference to the drawings. In the application example, an example will be described in which the knee trajectory information regarding the fifth example in FIGS. 10 to 11 is displayed on the screen of the terminal device. In the following application example, an example in which knee trajectory information (also referred to as display information) including visual information is superimposed and displayed on a video of a walking subject looking down from obliquely above will be described. The image of the subject may be an actual image or a virtual person (character) imitating the motion of the subject. Hereinafter, an example of displaying a character in a video will be described. The following display information may be generated by the knee trajectory information generation device 10 or may be generated by another device or system that has acquired the knee trajectory information.

### Application Example 1-1

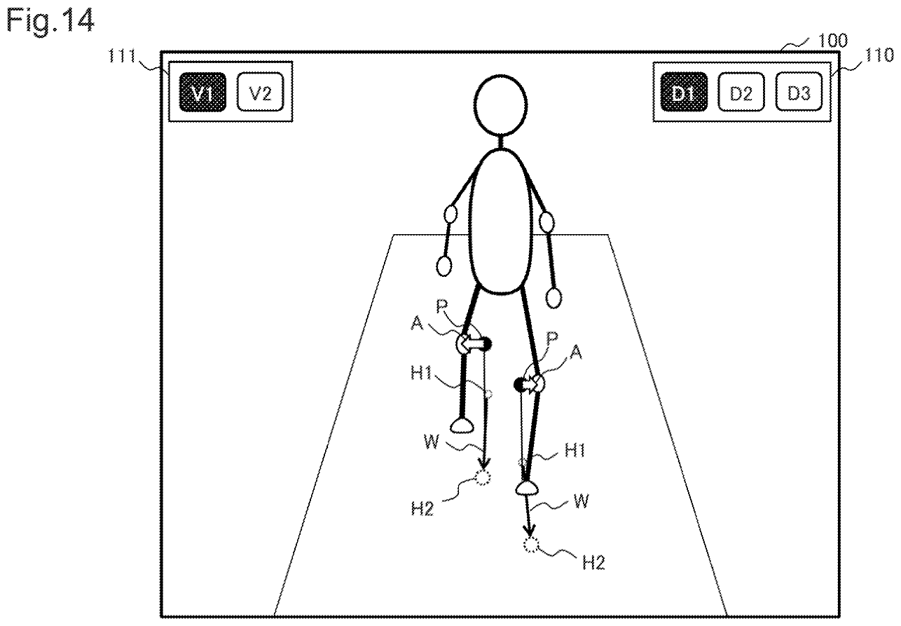

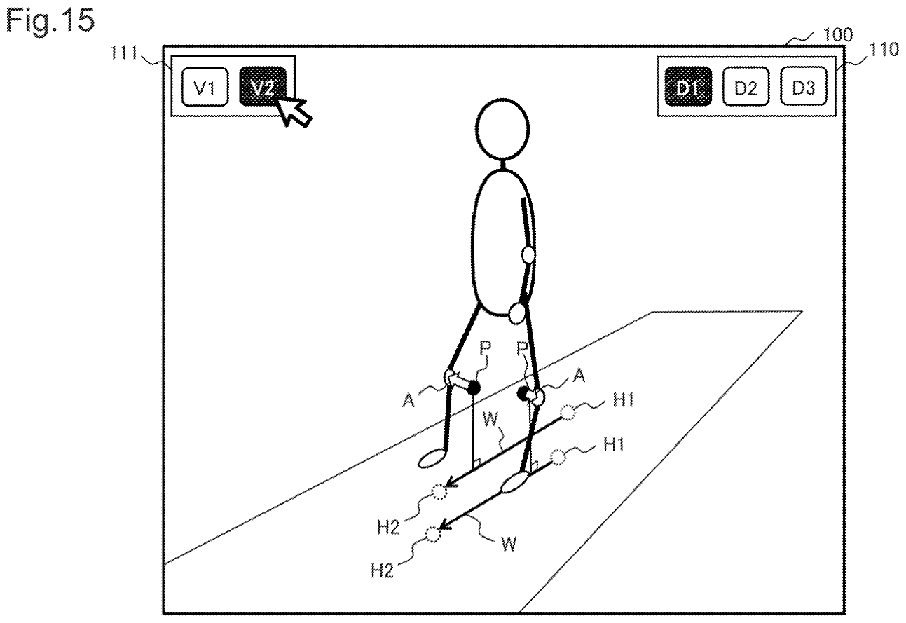

FIGS. 14 and 15 are conceptual diagrams relating to Application Example 1-1 relating to the knee trajectory information generation device 10. FIGS. 14 and 15 illustrate a first display pattern D1. In the first display pattern D1, the knee trajectory information in which the pin P is raised immediately above the first movement route W is displayed on the screen 100.

A display switching region 110 including a button for switching a display pattern is displayed at a position of an upper right corner of the screen 100. FIGS. 14 and 15 illustrate a state in which the first display pattern D1 is selected. A second display pattern D2 and a third display pattern D3 will be described later.

A viewpoint switching region 111 including a button for switching the viewpoint is displayed at the position of an upper left corner of the screen 100. In the viewpoint switching region 111, buttons for switching the viewpoint between a front viewpoint (first viewpoint V1) and a diagonally forward left viewpoint (second viewpoint V2) centering on the person (character) are displayed. The viewpoint corresponds to the viewpoint of the user viewing the screen 100. The display switching region 110 and the viewpoint switching region 111 are interface regions that receive a user's operation.

FIG. 14 illustrates an example in which a video of a person (character) viewed from a front viewpoint (first viewpoint V1) with the person (character) at the center is displayed on the screen 100. In FIG. 14, the first display pattern D1 is selected in the display switching region 110. In the viewpoint switching region 111, the first viewpoint V1 is selected. When viewed from the first viewpoint V1, it is easy to grasp the knee trajectory in the coronal plane. That is, when viewed from the first viewpoint V1, the behavior of the knee in the left-right direction (in the coronal plane) can be easily grasped.

FIG. 15 illustrates an example in which a video of a person (character) viewed from a diagonally forward left viewpoint (second viewpoint V2) centering on the person (character) is displayed on the screen 100. FIG. 15 illustrates a state in which the viewpoint has been switched from the first viewpoint V1 (FIG. 14) to the second viewpoint V2 in response to the selection of the second viewpoint V2 in the viewpoint switching region 111. As in the second viewpoint V2, when viewed from a viewpoint obliquely forward with respect to the traveling direction of the person (character), the knee trajectory can be easily three-dimensionally grasped.

### Application Example 1-2

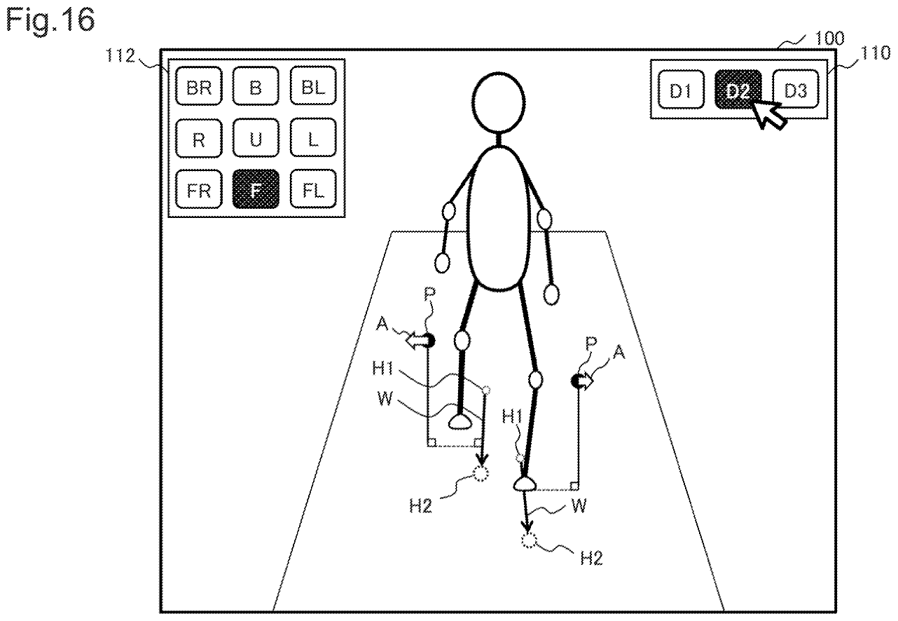

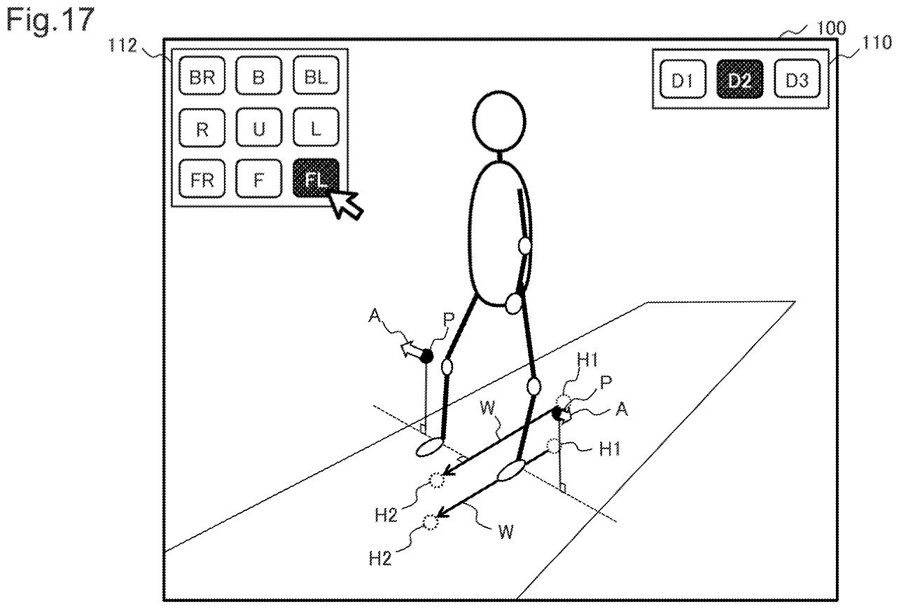

FIGS. 16 and 17 are conceptual diagrams relating to Application Example 1-2 relating to the knee trajectory information generation device 10. FIGS. 16 and 17 illustrate the second display pattern D2. In the second display pattern, the knee trajectory information in which the pin P is raised at a position away from the first movement route W in the coronal plane is displayed on the screen 100. For example, the pin P is displayed at a position away from the knee position by a multiple of the distance between the center of gravity of the body and the knee position.

A display switching region 110 including a button for switching a display pattern is displayed at a position of an upper right corner of the screen 100. FIG. 16 illustrates a state in which the display pattern is switched from the first display pattern D1 (FIG. 15) to the second display pattern D2 according to the selection of the second display pattern D2. In the second display pattern D2, the pin P is raised at a position away from the first movement route W in the coronal plane.

A viewpoint switching region 112 including a button for switching the viewpoint is displayed at the position of the upper left corner of the screen 100. For example, the viewpoint switching region 111 may be set to be switched to the viewpoint switching region 112 according to the switching of the display pattern from the first display pattern D1 to the second display pattern D2. The viewpoint switching region 112 may be set in the first display pattern D1, or the viewpoint switching region 111 may be set in the second display pattern D2.

In the viewpoint switching region 112, nine buttons for selecting the viewpoint are displayed. On the upper part of the viewpoint switching region 112, buttons for selecting a diagonally backward right viewpoint BR, a rear viewpoint B, and a diagonally backward left viewpoint BL with the person (character) as a center are displayed. In the middle part of the viewpoint switching region 112, buttons for selecting a right viewpoint R, an upper viewpoint U, and a left viewpoint L with the person (character) as a center are displayed. On the lower part of the viewpoint switching region 112, buttons for selecting a diagonally forward right viewpoint FR, a diagonally forward front viewpoint F, and a diagonally forward left viewpoint FL with the person (character) as a center are displayed. FIGS. 16 and 17 are examples, and the viewpoint that can be selected in the viewpoint switching region 112 is not limited. The display switching region 110 and the viewpoint switching region 112 are interface regions that receive a user's operation.

FIG. 16 illustrates an example in which a video of a person (character) viewed from the front viewpoint (viewpoint F) with the person (character) at the center is displayed on the screen 100. FIG. 16 illustrates a state in which the display pattern is switched from the first display pattern D1 (FIG. 15) to the second display pattern D2 according to the selection of the second display pattern D2 in the display switching region 110. In FIG. 16, the second display pattern D2 is selected in the display switching region 110. In the viewpoint switching region 112, the viewpoint F is selected. The viewpoint F is the same viewpoint as the first viewpoint V1 (FIG. 14). When viewed from the viewpoint F, it is easy to grasp the behavior of the knee in the left-right direction (coronal plane).

FIG. 17 illustrates an example in which a video of a person (character) viewed from the diagonally forward left viewpoint (viewpoint FL) centering on the person (character) is displayed on the screen 100. FIG. 17 illustrates a state in which the viewpoint is switched from the viewpoint F (FIG. 16) to the viewpoint FL in accordance with the selection of the viewpoint FL in the viewpoint switching region 112. The viewpoint FL is the same viewpoint as the second viewpoint V2 (FIG. 15). When viewed from the viewpoint FL, it is easy to grasp the three-dimensional knee trajectory.

In the present application example, the knee trajectory information with the pin P raised at a position away from the first movement route W in the coronal plane is displayed on the screen 100. According to the present application example, since the pin P and the arrow A do not overlap the person (character), it is easy to grasp the knee trajectory according to the change of the pin P and the arrow A.

### Application Example 1-3

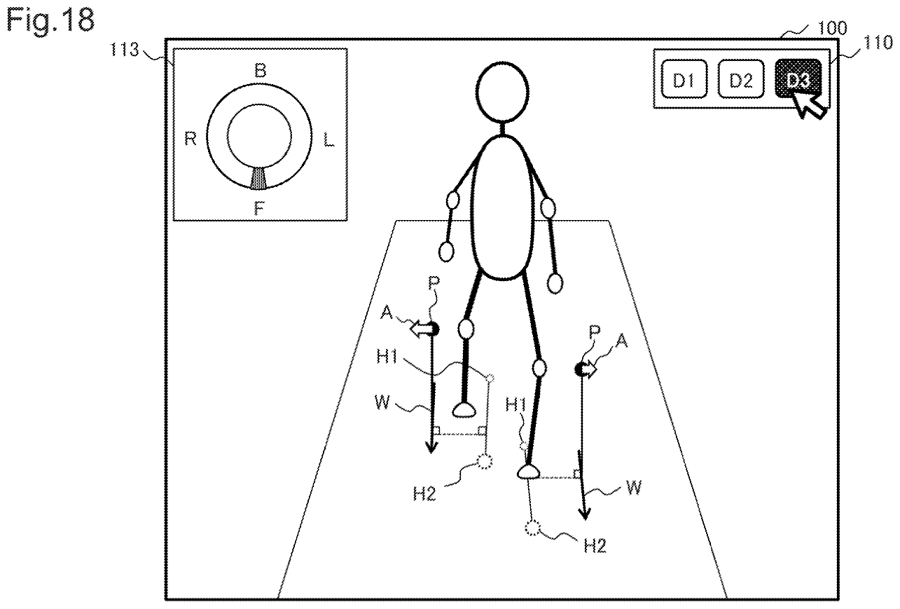

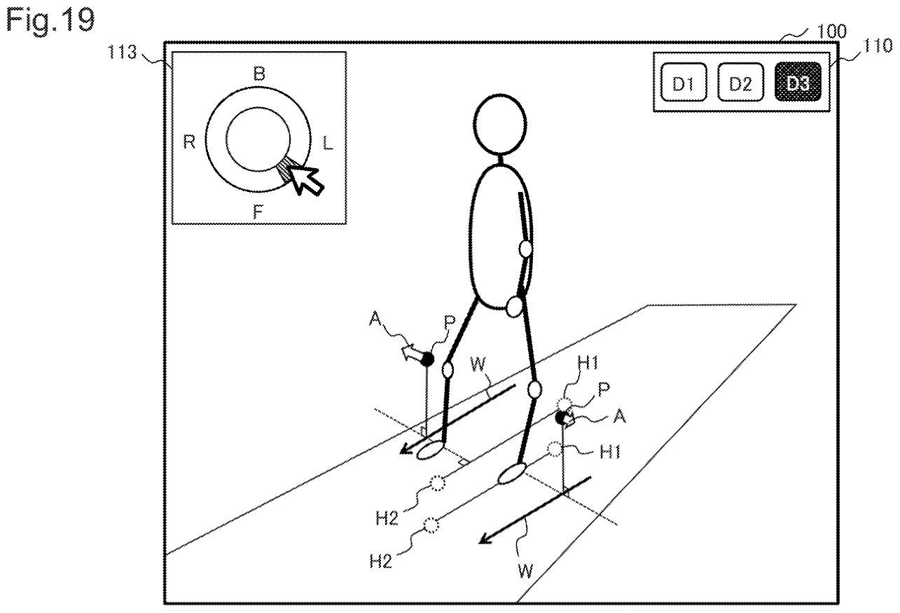

FIGS. 18 and 19 are conceptual diagrams relating to Application Example 1-3 relating to the knee trajectory information generation device 10. FIGS. 18 and 19 illustrate the third display pattern D3. In the third display pattern D3, the knee trajectory information in which the first movement route W is set at a position away from the person (character) is displayed on the screen 100. In the third display pattern D3, the knee trajectory information with the pin P raised immediately above the first movement route W is displayed. For example, the first movement route W is displayed at a position away from the knee position by a multiple of the distance between the center of gravity of the body and the knee position.

A display switching region 110 including a button for switching a display pattern is displayed at a position of an upper right corner of the screen 100. FIG. 18 illustrates a state in which the display pattern is switched from the second display pattern D2 (FIG. 17) to the third display pattern D3 according to the selection of the third display pattern D3. In the third display pattern D3, the first movement route W is displayed at a position away from the person (character). In the third display pattern D3, the pin P is raised immediately above the first movement route W.

A viewpoint switching region 113 including a user interface for switching the viewpoint is displayed at the position of the upper left corner of the screen 100. For example, the viewpoint switching region 112 may be set to be switched to the viewpoint switching region 113 according to the switching of the display pattern from the second display pattern D2 to the third display pattern D3. In the first display pattern D1 and the second display pattern D2, the viewpoint switching region 113 may be set.

In the viewpoint switching region 113, a circular user interface (hereinafter, referred to as a circle) for switching the viewpoint is displayed. A slider (hatching) for selecting a viewpoint is displayed on the circle. The slider moves along the circumference of the circle. A viewpoint is selected by adjusting the slider to a desired viewpoint position. The viewpoint selected through the user interface is a viewpoint centered on a person (character). In the examples of FIGS. 18 and 19, the position of the slider is associated with the viewpoint in the horizontal plane. In the circle, the lower side corresponds to the front viewpoint F, the right side corresponds to the left viewpoint L, the upper side corresponds to the rear viewpoint B, and the left side corresponds to the right viewpoint R. By moving the position of the slider along the circumference of the circle, the 360 degree viewpoint in the horizontal plane is switched. The display switching region 110 and the viewpoint switching region 113 are interface regions that receive a user's operation. FIGS. 18 and 19 are examples, and the viewpoint that can be selected in the viewpoint switching region 113 is not limited.

FIG. 18 illustrates an example in which a video of a person (character) viewed from the front viewpoint F with the person (character) at the center is displayed on the screen 100. FIG. 18 illustrates a state in which the display pattern is switched from the second display pattern D2 (FIG. 17) to the third display pattern D3 according to the selection of the third display pattern D3 in the display switching region 110. In FIG. 18, the third display pattern D3 is selected in the display switching region 110. In the viewpoint switching region 113, the viewpoint F is selected. The viewpoint F is the same viewpoint as the first viewpoint V1 (FIG. 14). When viewed from the viewpoint F, it is easy to grasp the behavior of the knee in the left-right direction (coronal plane).

FIG. 19 illustrates an example in which a video of a person (character) viewed from the diagonally forward left viewpoint between the front viewpoint F and the left viewpoint L with the person (character) at the center is displayed on the screen 100. FIG. 19 illustrates a state in which the viewpoint is switched from the viewpoint F (FIG. 18) on the front upper side to the diagonally forward left viewpoint with the person (character) as a center according to the operation of the slider of the circle in the viewpoint switching region 113. The diagonally forward left viewpoint with the person (character) as a center is the same viewpoint as the second viewpoint V2 (FIG. 15). When viewed from the diagonally forward left viewpoint with respect to the traveling direction of the person (character), it is easy to grasp a three-dimensional knee trajectory.

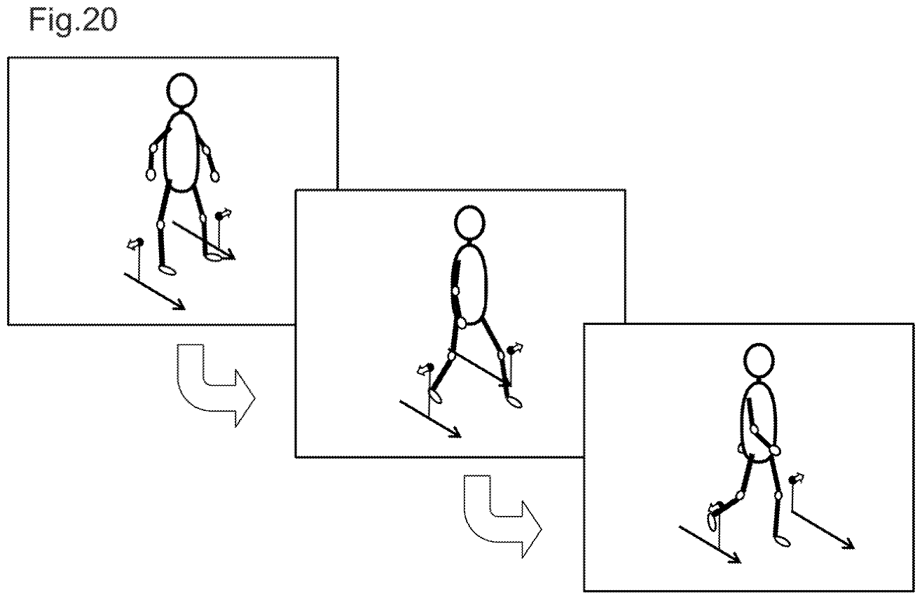

FIG. 20 is a conceptual diagram illustrating an example in which the first movement route W, the pin P, and the arrow A in Application Example 1-3 are displayed in association with a moving image. FIG. 20 illustrates a state of a person (character) as a center as viewed from the diagonally forward right viewpoint. FIG. 20 illustrates three frames extracted from among a plurality of frames included in a video related to walking of a person (character). The actual video is composed of more frames. In the three frames in FIG. 20, time (gait cycle) progresses from the upper left to the lower right. The video indicating the walking state of the person (character) changes according to the progress of time (gait cycle). The first movement route W, the pin P, and the arrow A change in accordance with the walking phase in the walking of the person (character).

In the present application example, the first movement route W is displayed at a position away from the person (character). In the present application example, the pin P is raised immediately above the first movement route W. In the present application example, the first movement route W, the pin P, and the arrow A do not overlap the person (character). Therefore, it is easy to grasp the change in the knee trajectory with respect to the first movement route according to the movement of the pin P and the arrow A moving immediately above the first movement route.

As described above, the knee trajectory information generation device according to the present example embodiment includes an acquisition unit, a first calculation unit, a second calculation unit, an information generation unit, and an output unit. The acquisition unit acquires walking data including time-series data of a foot position and a knee position of the subject. The first calculation unit calculates the first movement route connecting the start point and the end point of the gait cycle using the time-series data of the foot position included in the walking data. The second calculation unit calculates the second movement route corresponding to the locus of the knee position between the start point and the end point of the gait cycle using the time-series data of the knee position included in the walking data. The information generation unit calculates a difference between the first movement route and the second movement route. The information generation unit generates knee trajectory information including visual information corresponding to the calculated difference. The output unit outputs the generated knee trajectory information.

In the present example embodiment, the knee trajectory information including the visual information indicating the knee trajectory of the subject is generated. The visual information indicating the knee trajectory of the subject includes the behavior of the knee in the left-right direction. That is, according to the present example embodiment, it is possible to generate information regarding the knee trajectory including the behavior of the knee in the left-right direction.

In one aspect of the present example embodiment, the acquisition unit acquires the walking data of the subject with the continuous heel strikes as the start point and the end point of the gait cycle. The first calculation unit calculates the first movement route connecting the start point and the end point of the gait cycle in the horizontal plane. The second calculation unit calculates the second movement route corresponding to the locus of the knee position between the start point and the end point of the gait cycle in the horizontal plane. The information generation unit calculates a difference in the horizontal plane in association with the walking phase included in the gait cycle. The information generation unit generates the knee trajectory information including the visual information in which a difference is associated with the walking phase. According to the present aspect, it is possible to generate information regarding the knee trajectory including the behavior of the knee in the left-right direction with respect to the gait cycle with the continuous heel strike as the start point and the end point.

In one aspect of the present example embodiment, the information generation unit generates the visual information in which an arrow indicating a direction and a magnitude of a difference is associated with a walking phase included in a gait cycle. According to the present aspect, the behavior of the knee can be intuitively grasped according to the direction and the length of the arrow associated with the walking phase.

In one aspect of the present example embodiment, the information generation unit generates the visual information in which a sign obtained by combining the first sign indicating the height of the knee position and the second sign indicating the direction and magnitude of the difference is superimposed on a frame constituting a video indicating the walking state of the subject. According to the present aspect, it is possible to intuitively grasp the behavior of the knee according to the sign that changes in conjunction with the walking of the subject.

In one aspect of the present example embodiment, the information generation unit generates the visual information in which a sign is superimposed on the knee position of the subject displayed in the frame. According to the present aspect, it is possible to intuitively grasp the behavior of the knee according to the sign displayed at the knee position of the subject.

In one aspect of the present example embodiment, the information generation unit generates the visual information in which a sign is displayed at a position away from the subject displayed in the frame. According to the present aspect, it is possible to intuitively grasp the behavior of the knee according to the sign displayed at a position away from the subject.

In one aspect of the present example embodiment, the information generation unit generates the visual information in which a straight line indicating the first movement route and a sign are combined. According to the present aspect, it is easy to intuitively grasp the behavior of the knee in accordance with the walking phase of the subject.

In one aspect of the present example embodiment, the output unit outputs the knee trajectory information regarding the subject to the terminal device. The output unit displays the display information regarding the knee trajectory information on the screen of the terminal device. According to the present aspect, the behavior of the knee can be intuitively grasped by visually recognizing the display information displayed on the screen of the terminal device.

Knee osteoarthritis is a symptom in which inflammation or the like occurs in the knee joint due to degeneration of cartilage of the knee joint. Early detection and prevention of diseases are important for knee osteoarthritis. Regarding knee osteoarthritis, diagnosis is mainly performed subjectively by a doctor. Therefore, it is required to provide information supporting diagnosis by a doctor. In particular, the behavior of the knee at an initial stance period is important as one of diagnostic indices of knee osteoarthritis and the like. From the viewpoint of early detection and prevention, it is desirable that a sign of a disease related to the knee such as knee osteoarthritis is found early. According to the method of the present example embodiment, the behavior of the knee in the left-right direction can be clearly observed by the visual information indicating the knee trajectory of the subject. Therefore, according to the method of the present example embodiment, it is easy to find the slight lateral thrust according to the visual information displayed in the image. The method of the present example embodiment can also be applied to other than knee osteoarthritis as long as it is a symptom related to the knee. The method of the present example embodiment can be applied to various fields such as diagnosis of symptoms related to legs, rehabilitation, prevention of frailty, determination of falling risk, and the like.

### Second Example Embodiment

Next, a knee trajectory information generation device according to a second example embodiment will be described with reference to the drawings. The knee trajectory information generation device of the present example embodiment has a simplified configuration of a first knee trajectory information generation device.

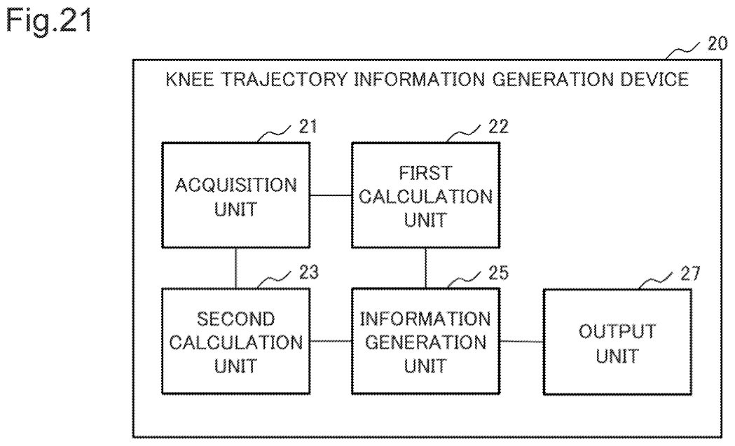

FIG. 21 is a block diagram illustrating an example of a configuration of a knee trajectory information generation device 20 according to the present example embodiment. The knee trajectory information generation device 20 includes an acquisition unit 21, a first calculation unit 22, a second calculation unit 23, an information generation unit 25, and an output unit 27.

The acquisition unit 21 acquires walking data including time-series data of the foot position and the knee position of the subject. The first calculation unit 22 calculates the first movement route connecting the start point and the end point of the gait cycle using the time-series data of the foot position included in the walking data. The second calculation unit 23 calculates the second movement route corresponding to the locus of the knee position between the start point and the end point of the gait cycle using the time-series data of the knee position included in the walking data. The information generation unit 25 calculates a difference between the first movement route and the second movement route. The information generation unit 25 generates knee trajectory information including visual information corresponding to the calculated difference. The output unit 27 outputs the generated knee trajectory information.

In the present example embodiment, the knee trajectory information including the visual information indicating the knee trajectory of the subject is generated. The visual information indicating the knee trajectory of the subject includes the behavior of the knee in the left-right direction. That is, according to the present example embodiment, it is possible to generate information regarding the knee trajectory including the behavior of the knee in the left-right direction.

(Hardware)

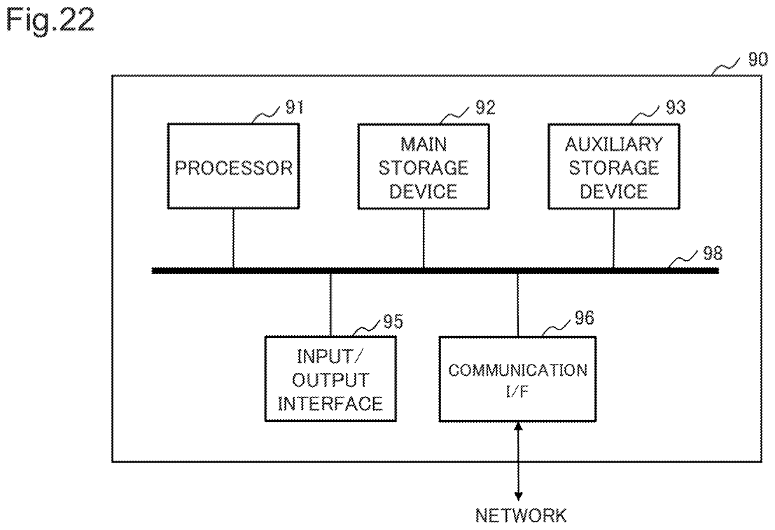

Here, a hardware configuration for executing the processing according to each example embodiment of the present disclosure will be described using an information processing device 90 (computer) of FIG. 22 as an example. The information processing device 90 in FIG. 22 is a configuration example for executing the processing of each example embodiment, and does not limit the scope of the present disclosure.

As illustrated in FIG. 22, the information processing device 90 includes a processor 91, a main storage device 92, an auxiliary storage device 93, an input/output interface 95, and a communication interface 96. In FIG. 22, the interface is abbreviated as an I/F. The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program (instruction) stored in the auxiliary storage device 93 or the like in the main storage device 92. For example, the program is a software program for executing the processing of each example embodiment. The processor 91 executes the program developed in the main storage device 92. The processor 91 executes the processing according to each example embodiment by executing the program.

The main storage device 92 has a region in which a program is developed. A program stored in the auxiliary storage device 93 or the like is developed in the main storage device 92 by the processor 91. The main storage device 92 is implemented by, for example, a volatile memory such as a dynamic random access memory (DRAM). A nonvolatile memory such as a magneto resistive random access memory (MRAM) may be configured and added as the main storage device 92.

The auxiliary storage device 93 stores various data such as programs. The auxiliary storage device 93 is implemented by a local disk such as a hard disk or a flash memory. Various data may be stored in the main storage device 92, and the auxiliary storage device 93 may be omitted.

The input/output interface 95 is an interface for connecting the information processing device 90 and a peripheral device. The communication interface 96 is an interface for connecting to an external system or device through a network such as the Internet or an intranet based on a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input information and settings. When a touch panel is used as the input device, a screen having a touch panel function serves as an interface. The processor 91 and the input device are connected via the input/output interface 95.

The information processing device 90 may be provided with a display device for displaying information. In a case where a display device is provided, the information processing device 90 may include a display control device (not illustrated) for controlling display of the display device. The display device may be connected to the information processing device 90 via the input/output interface 95.

The information processing device 90 may be provided with a drive device. The drive device mediates reading of data and a program stored in a recording medium and writing of a processing result of the information processing device 90 to the recording medium between the processor 91 and the recording medium (program recording medium). The information processing device 90 and the drive device are connected via an input/output interface 95.

The above is an example of the hardware configuration for enabling the processing according to each example embodiment of the present invention. The hardware configuration of FIG. 22 is an example of a hardware configuration for executing the processing of each example embodiment, and does not limit the scope of the present invention. A program for causing a computer to execute processing according to each example embodiment is also included in the scope of the present invention.

Further, a program recording medium in which the program according to each example embodiment is recorded is also included in the scope of the present invention. The recording medium can be implemented by, for example, an optical recording medium such as a compact disc (CD) or a digital versatile disc (DVD). The recording medium may be implemented by a semiconductor recording medium such as a universal serial bus (USB) memory or a secure digital (SD) card. The recording medium may be implemented by a magnetic recording medium such as a flexible disk, or another recording medium. When a program executed by the processor is recorded in a recording medium, the recording medium is associated to a program recording medium.

The components of each example embodiment may be arbitrarily combined. The components of each example embodiment may be implemented by software. The components of each example embodiment may be implemented by a circuit.

The previous description of embodiments is provided to enable a person skilled in the art to make and use the present invention. Moreover, various modifications to these example embodiments will be readily apparent to those skilled in the art, and the generic principles and specific examples defined herein may be applied to other embodiments without the use of inventive faculty. Therefore, the present invention is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A knee trajectory information generation device comprising:
at least one memory storing instructions; and
at least one processor connected to the at least one memory and configured to execute the instructions to:
acquire walking data including time-series data of a foot position and a knee position of a subject;
calculate a first movement route connecting a start point and an end point of a gait cycle by using time-series data of the foot position included in the walking data;
calculate a second movement route corresponding to a trajectory of the knee position between the start point and the end point of the gait cycle by using time-series data of the knee position included in the walking data;
calculate a difference between the first movement route and the second movement route and generate knee trajectory information including visual information corresponding to the calculated difference; and
output the generated knee trajectory information.

2. The knee trajectory information generation device according to claim 1, wherein
the at least one processor is configured to execute the instructions to
acquire the walking data of the subject with continuous heel strike as the start point and the end point of the gait cycle,
calculate the first movement route connecting the start point and the end point of the gait cycle in a horizontal plane,
calculate the second movement route corresponding to the trajectory of the knee position between the start point and the end point of the gait cycle in the horizontal plane,
calculate the difference in the horizontal plane in association with a walking phase included in the gait cycle, and
generate the knee trajectory information including the visual information in which the difference is associated with the walking phase.

3. The knee trajectory information generation device according to claim 1, wherein
the at least one processor is configured to execute the instructions to
generate the visual information in which an arrow indicating a direction and a magnitude of the difference is associated with a walking phase included in the gait cycle.

4. The knee trajectory information generation device according to claim 1, wherein
the at least one processor is configured to execute the instructions to
generate the visual information in which a sign obtained by combining a first sign indicating a height of the knee position and a second sign indicating a direction and a magnitude of the difference is superimposed on a frame constituting a video indicating a walking state of the subject.

5. The knee trajectory information generation device according to claim 4, wherein
the at least one processor is configured to execute the instructions to
generate the visual information in which the sign is superimposed on the knee position of the subject displayed in the frame.

6. The knee trajectory information generation device according to claim 4, wherein
the at least one processor is configured to execute the instructions to:
generate the visual information in which the sign is displayed at a position away from the subject displayed in the frame.

7. The knee trajectory information generation device according to claim 4, wherein
the at least one processor is configured to execute the instructions to
generate the visual information in which a straight line indicating the first movement route and the sign are combined.

8. The knee trajectory information generation device according to claim 1, wherein
the at least one processor is configured to execute the instructions to
output the knee trajectory information regarding the subject to a terminal device; and
display display information regarding the knee trajectory information on a screen of the terminal device.

9. A knee trajectory information generation method for causing a computer to execute:
acquiring walking data including time-series data of a foot position and a knee position of a subject;
calculating a first movement route connecting a start point and an end point of a gait cycle by using time-series data of the foot position included in the walking data;
calculating a second movement route corresponding to a trajectory of the knee position between the start point and the end point of the gait cycle by using time-series data of the knee position included in the walking data;
calculating a difference between the first movement route and the second movement route and generating knee trajectory information including visual information corresponding to the calculated difference; and
outputting the generated knee trajectory information.

10. A non-transitory recording medium having stored therein a program for causing a computer to execute:
acquiring walking data including time-series data of a foot position and a knee position of a subject;
calculating a first movement route connecting a start point and an end point of a gait cycle by using time-series data of the foot position included in the walking data;
calculating a second movement route corresponding to a trajectory of the knee position between the start point and the end point of the gait cycle by using time-series data of the knee position included in the walking data;
calculating a difference between the first movement route and the second movement route and generating knee trajectory information including visual information corresponding to the calculated difference; and
outputting the generated knee trajectory information.
